(19) United States
(12) Patent Application Publication
(10) Pub. No.: US 2011/0027870 A1
(43) Pub. Date: Feb. 3, 2011

(54) BIOMASS CARRIERS, METHOD AND APPARATUS FOR MANUFACTURE THEREOF AND FLUID TREATMENT SYSTEMS AND METHODS UTILIZING SAME

(75) Inventors: Nir Assulin, Hibat-Zion (IL); Ronen Itzhak Shechter, Ramat Tivon (IL); Tamar Arbel, Karmiel (IL)

Correspondence Address: SUGHRUE MION, PLLC, 2100 PENNSYLVANIA AVENUE, N.W., SUITE 800, WASHINGTON, DC, 20037, US

(73) Assignee: AQWISE-WISE WATER TECHNOLOGIES LTD., Herzilia (IL)

(21) Appl. No.: 12/919,137

(22) PCT Filed: Feb. 24, 2009
(86) PCT No.: PCT/IL09/00209

§ 371 Date: Oct. 15, 2010

Related U.S. Application Data
(60) Provisional application No. 61/031,076, filed on Feb. 25, 2008.

(51) Int. Cl. C12M 1/00; B01J 19/30; C02F 3/10; B32B 37/16

(52) U.S. Cl. 435/266; 435/299.1; 428/35.7; 210/150; 210/615; 156/296; 156/349

(57) ABSTRACT

A biomass carrier including a multiplicity of generally elongate plastic biomass attachment strips joined to each other at least one location therealong, the multiplicity of generally elongate plastic biomass attachment strips being mutually arranged to define a generally deformable biomass carrier including generally outer disposed biomass attachment strips and generally inner disposed biomass attachment strips. Apparatus and methods for manufacture of the biomass carrier as well as fluid treatment systems and methods using the biomass carrier are also disclosed.

### REFERENCE TO RELATED APPLICATIONS

Reference is made to U.S. Provisional Patent Application Ser. No. 61/031,076, filed Feb. 25, 2008, the disclosure of which is hereby incorporated by reference and priority of which is hereby claimed pursuant 35 CFR 1.78(a)(4) and (5)(i).

Reference is also made to U.S. Pat. No. 6,616,845; U.S. Pat. No. 6,726,838; published PCT Patent Application WO 02096806; published PCT Patent Application WO 2007029256 and published PCT Patent Application WO 2008018077, the disclosures of which are hereby incorporated by reference.

### FIELD OF THE INVENTION

The present invention relates to fluid treatment generally and more particularly to fluid treatment systems and methodologies employing biomass carriers and to biomass carriers useful therein.

### BACKGROUND OF THE INVENTION

The following publications are believed to represent the current state of the art:

U.S. Pat. Nos.:

| | | | | |
|---|---|---|---|---|
| 4,310,437; | 4,373,024; | 4,507,546; | 4,960,540; | 5,108,655; |
| 5,783,089; | 5,827,453; | 5,928,493; | 6,207,722; | 6,534,550; |
| 6,616,845; | 6,660,164; | 6,689,271; | 6,726,838; | 6,960,304; |
| 6,962,653; | 7,001,519; | 4,279,753; | 6,110,389; | 4,322,296; |
| 4,620,929; | 6,565,750; | 3,506,125; | 4,810,377; | 5,080,793; |
| 5,458,779; | 4,188,289; | 3,133,017; | 1,790,975; | 2,709,128; |
| 5,779,886; | 5,490,934; | 6,726,838; | 3,957,931; | 4,179,366; |
| 4,333,893; | 4,385,988; | 4,522,767; | 4,537,731; | 4,814,085; |
| 4,814,125; | 4,842,920; | 4,985,182; | 4,999,103; | 5,168,058; |
| 5,192,442; | 5,200,081; | 5,217,616; | 5,429,740; | 5,486,292; |
| 5,543,039; | 5,558,763; | 5,783,066; | 5,783,069; | 5,871,674; |
| 5,902,484; | 5,948,262; | 5,962,309; | 5,980,738; | 5,981,272; |
| 5,985,148; | 5,993,650; | 6,015,497; | 6,063,268; | 6,077,424; |
| 6,126,829; | 6,136,194; | 6,156,204; | 6,210,578; | 4,137,171; |
| 4,045,344; | 3,133,017; | 4,394,268; | 4,521,311; | 5,554,289; |
| 4,566,971; | 4,820,415; | 6,063,863; | 4,839,053; | 4,599,174; |
| 4,231,863; | 4,374,730; | 5,030,353; | 5,202,027; | 5,698,094; |

| | | |
|---|---|---|
| 4,256,573; | 4,454,038 | and 1,498,360; |

Published U.S. patent application Nos.: 2003/0087969 and 2004/0089592;

Non-US Patent Publication Nos.: DE 39 16 520; FR 2 707 183; ES 2 064 083; EP 0 575 314; EP 0 750 59; EP 1 340 720; WO 95/33695; WO 91/11396 and WO 95/25072

Other Publications:
1. “Biological treatment of highly foaming pharmaceutical wastewater by modified bubble-column under mechanical foam control”, K. Yamagiwa, M. Yoshida, A. Ohkawa and S. Takesono, Water Science & Technology, VoI 42 No 3-4, pp 331-337, IWA Publishing 2000;
2. “Performance characteristics of mechanical foam-breakers fitted to a stirred-tank reactor”, Takesono S., Onodera M., Yoshida M., Yamagiwa K., Ohkawa A., Journal of Chemical Technology & Biotechnology, Volume 78, Number 1, January 2003, pp. 48-55(8);
3. Database WPI Week 198730 Derwent Publications Ltd., London, GB; An 1987-209691, XP002381758 & JP 62 136296, 19 Jun. 1987, an abstract.
4. Database WPI Week 199901 Derwent Publications Ltd., London, GB; An 1999-003480, XP002381759 & JP 10 277536, 20 Oct. 1998, an abstract.
5. Patent Abstracts of Japan Vol. 1996, no. 2, 29 Feb. 1996 & JP 07 275886, 24 Oct. 1995.

### SUMMARY OF THE INVENTION

The present invention seeks to provide improved biomass carriers, methods and apparatus for manufacture thereof and fluid treatment systems and methods using such carriers.

There is thus provided in accordance with a preferred embodiment of the present invention a biomass carrier including a multiplicity of generally elongate plastic biomass attachment strips joined to each other at least one location therealong, the multiplicity of generally elongate plastic biomass attachment strips being mutually arranged to define a generally deformable biomass carrier including generally outer disposed biomass attachment strips and generally inner disposed biomass attachment strips.

Preferably, the multiplicity of generally elongate plastic biomass attachment strips has a ratio of effective biomass attachment surface area to weight of between 10 to 30 m²/Kg carriers. More preferably, the multiplicity of generally elongate plastic biomass attachment strips has a ratio of effective biomass attachment surface area to weight of between 15 to 25 m²/Kg carriers. Alternatively, the multiplicity of generally elongate plastic biomass attachment strips has a ratio of effective biomass attachment surface area to overall volume of between 600 to 1500 m²/m³ carriers.

In accordance with a preferred embodiment of the present invention, the multiplicity of generally elongate plastic biomass attachment strips has a ratio of effective biomass attachment surface area to overall volume of between 900 to 1200 m²/m³ carriers.

Preferably, the multiplicity of generally elongate plastic biomass attachment strips includes generally elongate plastic biomass attachment strips having at least two different lengths.

In accordance with a preferred embodiment of the present invention, the multiplicity of generally elongate plastic biomass attachment strips are joined to each other at two locations.

Preferably, the multiplicity of generally elongate plastic biomass attachment strips are joined to each other at their respective ends.

In accordance with an embodiment of the present invention, the multiplicity of generally elongate plastic biomass attachment strips are crimped.

Preferably, the multiplicity of generally elongate plastic biomass attachment strips are welded to each other at their respective ends.

There is also provided in accordance with a preferred embodiment of the invention a method for manufacturing biomass carriers, the method including providing generally elongate plastic biomass attachment strips and joining the generally elongate plastic biomass attachment strips at least one location therealong, thereby to define generally deformable biomass carriers.

Preferably, the joining includes joining the generally elongate plastic biomass attachment strips at spaced locations therealong, thereby to define multiple generally deformable biomass carriers.

In accordance with a preferred embodiment of the present invention, the method includes aligning the generally elongate plastic biomass attachment strips of at least two different lengths at their respective ends, thereby causing at least some of the generally elongate plastic biomass attachment strips, when joined at both of their respective ends, to be under tension and some of the generally elongate plastic biomass attachment strips to be under compression.

Preferably, the method also includes aligning the generally elongate plastic biomass attachment strips of at least two different lengths at their respective ends, thereby causing at least some of the generally elongate plastic biomass attachment strips, when joined at both of their respective ends, to be at least partially outwardly bowed.

The method also may include crimping the generally elongate plastic biomass attachment strips.

In accordance with an embodiment of the invention, at least the joining step is carried out in propinquity to a fluid treatment system.

Preferably, the method also includes dispensing multiple ones of the biomass carriers into the fluid treatment system as they are manufactured.

In accordance with a preferred embodiment of the present invention, the method also includes compressing multiple ones of the biomass carriers, shipping the multiple ones of the biomass carriers following compression thereof to a fluid treatment system site and decompressing the multiple ones of the biomass carriers at the fluid treatment system site.

Preferably volume compression of at least a factor of 3 is realized.

There is also provided in accordance with a preferred embodiment of the present invention, apparatus for manufacturing biomass carriers, the apparatus comprising a positioner for positioning a multiplicity of generally elongate plastic biomass attachment strips and a binder, joining the plurality of generally elongate plastic biomass attachment strips downstream of the positioner at least one location therealong, thereby to define generally deformable biomass carriers.

Preferably, the binder is operative for joining the generally elongate plastic biomass attachment strips at predetermined spaced locations therealong.

In accordance with a preferred embodiment of the present invention, the generally elongate plastic biomass attachment strips include generally elongate plastic biomass attachment strips of at least two different lengths.

Preferably, the apparatus also includes an aligner for aligning the generally elongate plastic biomass attachment strips of at least two different lengths at their respective ends, thereby causing at least some of the generally elongate plastic biomass attachment strips, when joined at both of their respective ends, to be under tension and some of the generally elongate plastic biomass attachment strips to be under compression.

Preferably, the aligner is operative for aligning the generally elongate plastic biomass attachment strips of at least two different lengths at their respective ends, thereby causing at least some of the generally elongate plastic biomass attachment strips, when joined at both of their respective ends, to be at least partially outwardly bowed.

In accordance with a preferred embodiment of the present invention, the apparatus also includes a crimper for crimping the generally elongate plastic biomass attachment strips.

In accordance with an embodiment of the present invention, at least the binder is located in propinquity to a fluid treatment system.

In such an embodiment, preferably the apparatus also includes a dispenser, dispensing multiple ones of the biomass carriers into the fluid treatment system as they are manufactured.

In accordance with another embodiment of the present invention, the apparatus includes a compressor for compressing multiple ones of the biomass carriers. Preferably, the compressor provides volume compression of the biomass carriers of at least a factor of 3.

Additionally in accordance with a preferred embodiment of the present invention there is provided a fluid treatment system including at least one bioreactor having a fluid inlet for receiving fluid to be treated and a fluid outlet for providing treated fluid and a plurality of biomass carriers located within the at least one bioreactor, at least some of the plurality of biomass carriers each comprising a multiplicity of generally elongate plastic biomass attachment strips joined to each other at least one location therealong, the multiplicity of generally elongate plastic biomass attachment strips being mutually arranged to define a generally deformable biomass carrier including generally outer disposed biomass attachment strips and generally inner disposed biomass attachment strips.

Preferably, the bioreactor operates in at least one of an aerobic, an anoxic and an anaerobic mode of operation.

The bioreactor may be operative for treating a liquid or a gas.

Preferably the system includes a mixer operative to create turbulence between the fluid and the biomass carriers and or at least one diffuser operative to create turbulence between the fluid and the biomass carriers and to supply oxygen to the biomass attached on the biomass carriers.

There is additionally provided in accordance with a preferred embodiment of the present invention a fluid treatment method including providing a bioreactor having located therewithin a plurality of biomass carriers, at least some of the plurality of biomass carriers each comprising a multiplicity of generally elongate plastic biomass attachment strips joined to each other at least one location therealong, the multiplicity of generally elongate plastic biomass attachment strips being mutually arranged to define a generally deformable biomass carrier including generally outer disposed biomass attachment strips and generally inner disposed biomass attachment strips, receiving at a fluid inlet of the bioreactor, fluid to be treated and providing treated fluid at a fluid outlet of the bioreactor.

### BRIEF DESCRIPTION OF THE DRAWINGS

The present invention will be understood and appreciated more fully from the following detailed description, taken in conjunction with the drawings in which:

FIG. 1A is a simplified plan view illustration of a biomass carrier constructed and operative in accordance with a preferred embodiment of the present invention;

FIG. 1B is a simplified plan view illustration of a biomass carrier constructed and operative in accordance with another preferred embodiment of the present invention;

FIG. 2A is a simplified plan view illustration of a biomass carrier constructed and operative in accordance with a further preferred embodiment of the present invention;

FIG. 2B is a simplified plan view illustration of a biomass carrier constructed and operative in accordance with yet another preferred embodiment of the present invention;

FIG. 3A is a simplified plan view illustration of a biomass carrier constructed and operative in accordance with a still further preferred embodiment of the present invention;

FIG. 3B is a simplified plan view illustration of a biomass carrier constructed and operative in accordance with yet a further preferred embodiment of the present invention;

FIGS. 4A & 4B are simplified respective exploded view and assembled illustrations of apparatus for the manufacture of a biomass carrier of the type shown in FIG. 1A in accordance with a preferred embodiment of the present invention;

FIGS. 5A-5Q are simplified illustrations of the manufacture of a biomass carrier of the type shown in FIG. 1A using the apparatus of FIGS. 4A & 4B;

FIG. 6A is a simplified illustration of compression of the biomass carriers of the type shown in FIG. 1A in accordance with a preferred embodiment of the present invention;

FIG. 6B is a simplified illustration of compression of the biomass carriers of the type shown in FIG. 1A in accordance with another preferred embodiment of the present invention;

FIG. 7 is a simplified illustration of on site manufacture and introduction of biomass carriers of the type shown in FIG. 1A in a water treatment facility;

FIG. 8 is a simplified illustration of an embodiment of water treatment system employing biomass carriers of the type shown in FIG. 1A; and

FIGS. 9A & 9B are simplified illustrations of an embodiment of a gas treatment system employing biomass carriers of the type shown in FIG. 1A;

### DETAILED DESCRIPTION OF PREFERRED EMBODIMENTS

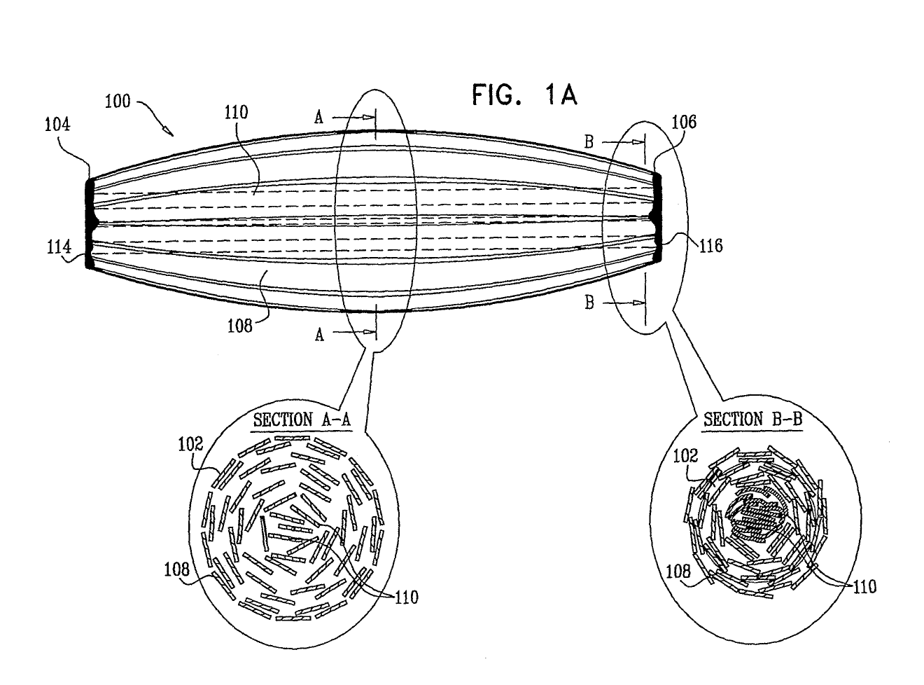

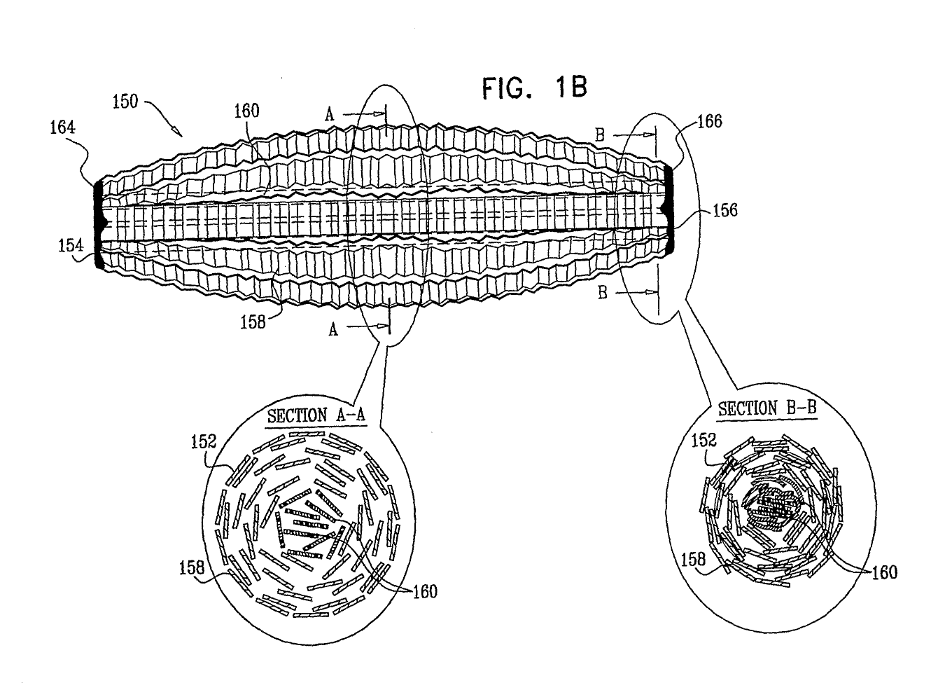

Reference is now made to FIG. 1A, which is a simplified plan view illustration of a biomass carrier 100 constructed and operative in accordance with a preferred embodiment of the present invention. As seen in FIG. 1A, biomass carrier 100 comprises a multiplicity of generally elongate plastic biomass attachment strips 102 joined to each other at respective opposite ends 104 and 106 thereof. In accordance with a preferred embodiment of the present invention, the multiplicity of generally elongate plastic biomass attachment strips are mutually arranged to define a generally deformable biomass carrier volume including generally outer disposed biomass attachment strips 108 and generally inner disposed biomass attachment strips 110.

In accordance with a preferred embodiment of the present invention, generally inner disposed biomass attachment strips 110 are shorter than generally outer disposed biomass attachment strips 108. Preferably all of the biomass attachment strips 102 are formed of a material which is resistant to axial compression and thus when the respective opposite ends 104 and 106 of generally inner disposed biomass attachment strips 110 and generally outer disposed biomass attachment strips 108 are joined, generally inner disposed biomass attachment strips 110 are tensioned and generally outer disposed biomass attachment strips 108 are caused to bow outward, as seen in FIG. 1A.

Preferably the ends 104 and 106 of the biomass attachment strips 102 are joined by welding, such as heat welding or ultrasonic welding, which produces respective generally sealed end portions 114 and 116.

A preferred material for the biomass attachment strips is high density polyethylene and preferred dimensions of the strips are as follows:

strips 108—length 50 mm, width 2-3 mm and thickness 20-60 microns

strips 110—length 47 mm, width 2-3 mm and thickness 20-60 microns.

Preferred overall dimensions of the biomass carrier of FIG. 1A are as follows:

length including end portions 114 and 116—50 mm, maximum width 15-20 mm.

Reference is now made to FIG. 1B, which is a simplified plan view illustration of a biomass carrier 150 constructed and operative in accordance with another preferred embodiment of the present invention. As seen in FIG. 1B, biomass carrier 150 comprises a multiplicity of generally elongate plastic biomass attachment strips 152 joined to each other at respective opposite ends 154 and 156 thereof. In accordance with a preferred embodiment of the present invention, the multiplicity of generally elongate crimped plastic biomass attachment strips are mutually arranged to define a generally deformable biomass carrier volume including generally outer disposed crimped biomass attachment strips 158 and generally inner disposed non-crimped biomass attachment strips 160.

In accordance with a preferred embodiment of the present invention, generally inner disposed biomass attachment strips 160 are shorter than generally outer disposed biomass attachment strips 158. Preferably at least the inner biomass attachment strips 160 are formed of a material which is resistant to axial compression and thus when the respective opposite ends 154 and 156 of generally inner disposed biomass attachment strips 160 and generally outer disposed crimped biomass attachment strips 158 are joined, generally inner disposed biomass attachment strips 160 are tensioned and generally outer disposed crimped biomass attachment strips 158 are caused to bow outward, as seen in FIG. 1A. The crimped configuration of outer disposed biomass attachment strips 158 increases their surface area.

Preferably, the ends 154 and 156 of the biomass attachment strips 152 are joined by welding, such as heat welding or ultrasonic welding, which produces respective generally sealed end portions 164 and 166.

A preferred material for the biomass attachment strips is high density polyethylene and preferred dimensions of the strips are as follows:

crimped strips 158—untensioned length 50 mm, width 2-3 mm and thickness 20-60 microns

uncrimped strips 160—length 47 mm, width 2-3 mm and thickness 20-60 microns.

Preferred overall dimensions of the biomass carrier of FIG. 1B are as follows:

length including end portions 164 and 166—50 mm, maximum width 15-20 mm.

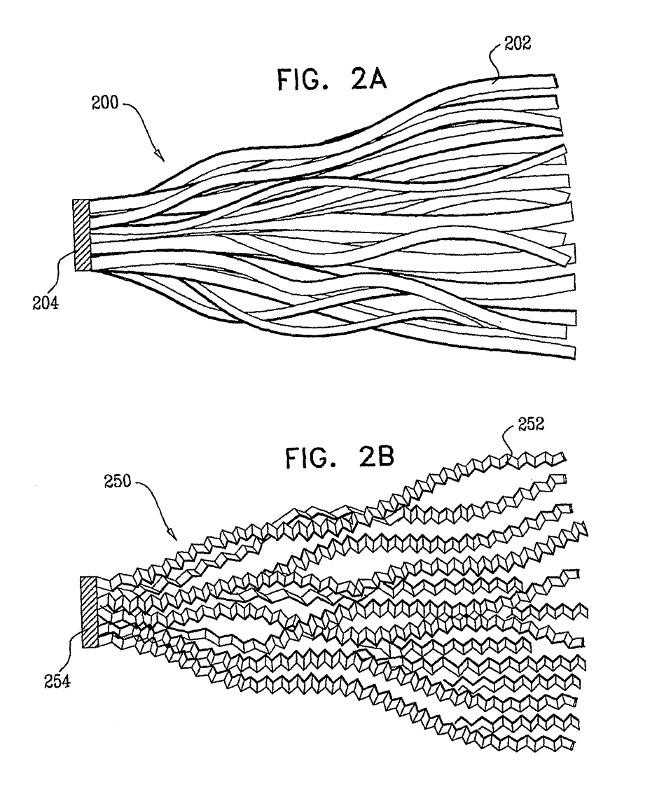

Reference is now made to FIG. 2A, which is a simplified plan view illustration of a biomass carrier 200 constructed and operative in accordance with yet another preferred embodiment of the present invention. As seen in FIG. 2A, biomass carrier 200 comprises a multiplicity of generally elongate plastic biomass attachment strips 202 joined to each other at a single end 204. In accordance with a preferred embodiment of the present invention, the multiplicity of generally elongate plastic biomass attachment strips are mutually arranged to define a generally deformable biomass carrier volume.

Preferably the ends of the biomass attachment strips 202 are joined by welding, such as heat welding or ultrasonic welding.

A preferred material for the biomass attachment strips 202 is high density polyethylene and preferred dimensions of the strips 202 is as follows:

length 50 mm, width 2-3 mm and thickness 20-60 microns

A preferred overall length of the biomass carrier of FIG. 2A, including the welded end portion thereof, is 50 mm.

Reference is now made to FIG. 2B, which is a simplified plan view illustration of a biomass carrier 250 constructed and operative in accordance with yet another preferred embodiment of the present invention. As seen in FIG. 2B, biomass carrier 250 comprises a multiplicity of generally elongate crimped plastic biomass attachment strips 252 joined to each other at a single end 254. In accordance with a preferred embodiment of the present invention, the multiplicity of generally elongate plastic crimped biomass attachment strips are mutually arranged to define a generally deformable biomass carrier volume.

Preferably the ends of the crimped biomass attachment strips 252 are joined by welding, such as heat welding or ultrasonic welding.

A preferred material for the biomass attachment strips 252 is high density polyethylene and preferred dimensions of the strips 252 is as follows:

length 50 mm, width 2-3 mm and thickness 20-60 microns

A preferred overall length of the biomass carrier of FIG. 2B, including the welded end portion thereof, is 50 mm.

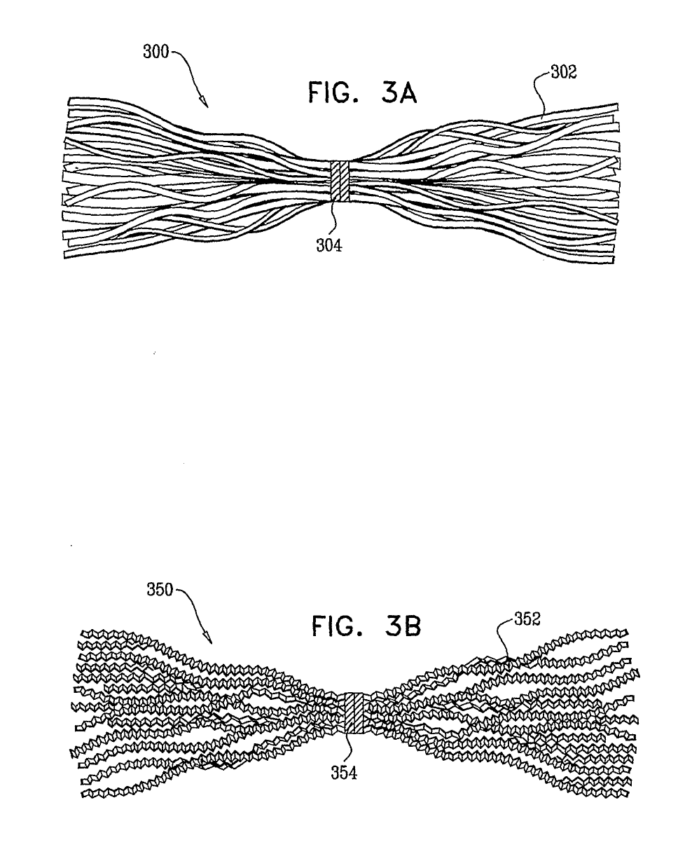

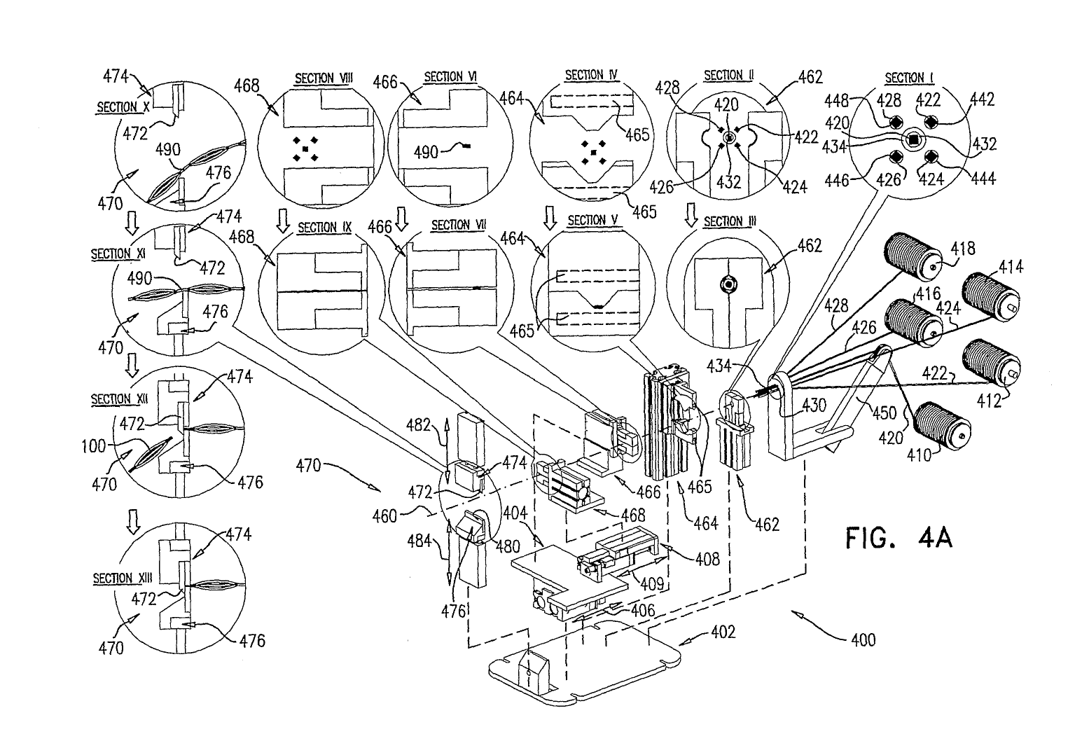

Reference is now made to FIG. 3A, which is a simplified plan view illustration of a biomass carrier 300 constructed and operative in accordance with yet another preferred embodiment of the present invention. As seen in FIG. 3A, biomass carrier 300 comprises a multiplicity of generally elongate plastic biomass attachment strips 302 joined to each other at a single location 304 intermediate the ends thereof. In accordance with a preferred embodiment of the present invention, the multiplicity of generally elongate plastic biomass attachment strips are mutually arranged to define a generally deformable biomass carrier volume.

Preferably the biomass attachment strips 302 are joined at location 304 by welding, such as heat welding or ultrasonic welding.

A preferred material for the biomass attachment strips 302 is high density polyethylene and preferred dimensions of the strips 302 is as follows:

length 50 mm, width 2-3 mm and thickness 20-60 microns.

A preferred overall length of the biomass carrier of FIG. 3A, including the welded end portion thereof, is 50 mm.

Reference is now made to FIG. 38, which is a simplified plan view illustration of a biomass carrier 350 constructed and operative in accordance with yet another preferred embodiment of the present invention. As seen in FIG. 3B, biomass carrier 350 comprises a multiplicity of generally elongate crimped plastic biomass attachment strips 352 joined to each other at a single location 354 intermediate the ends thereof. In accordance with a preferred embodiment of the present invention, the multiplicity of generally elongate plastic crimped biomass attachment strips are mutually arranged to define a generally deformable biomass carrier volume.

Preferably the crimped biomass attachment strips 352 are joined at location 354 by welding, such as heat welding or ultrasonic welding.

A preferred material for the biomass attachment strips 352 is high density polyethylene and preferred dimensions of the strips 352 is as follows:

length 50 mm, width 2-3 mm and thickness 20-60 microns

A preferred overall length of the biomass carrier of FIG. 3B, including the welded end portion thereof, is 50 mm.

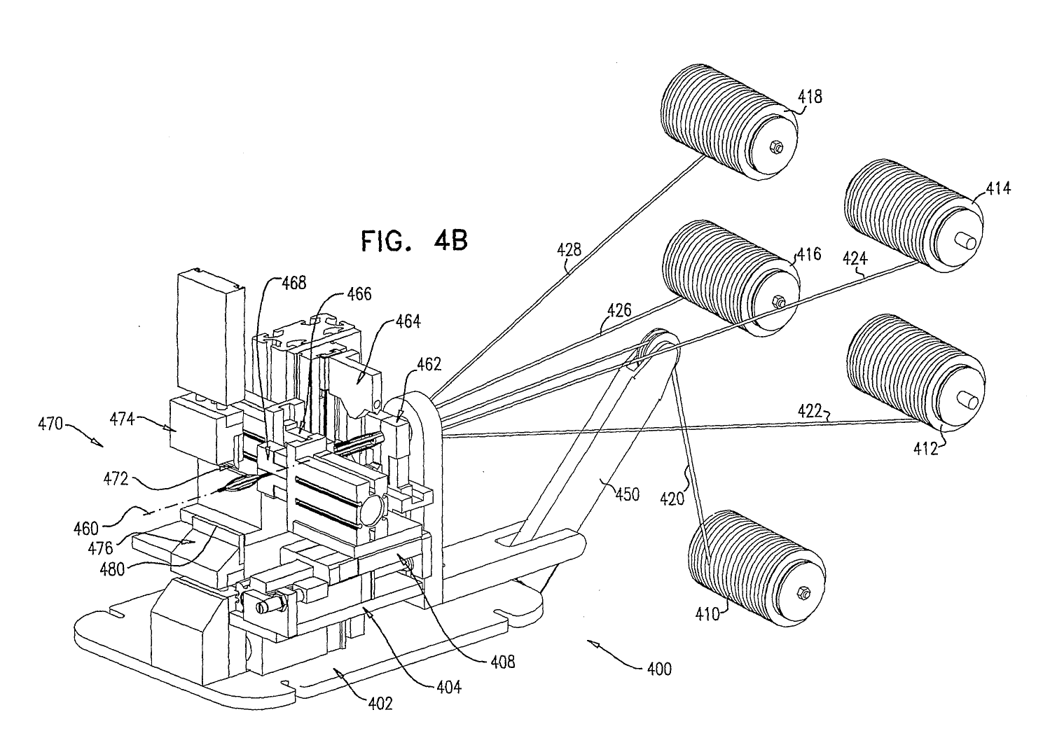

Reference is now made to FIGS. 4A & 4B, which are simplified illustrations of apparatus 400 for the manufacture of a biomass carrier of the type shown in FIG. 1A in accordance with a preferred embodiment of the present invention.

Apparatus 400 comprises a chassis 402 onto which is mounted a displaceable table assembly 404, which is arranged for reciprocal displacement along an X-axis indicated by arrows 406. A support assembly 408 is mounted onto displaceable table assembly 404 and is arranged for reciprocal displacement along the X-axis, as indicated by arrows 409.

Preferably, five supply rollers 410, 412, 414, 416 and 418 supply respective stacks 420, 422, 424, 426 and 428 of generally elongate plastic biomass attachment strips 102 (FIGS. 1A & 1B) to an arranging assembly 430. Each stack of generally elongate plastic strips 102 typically includes approximately ten strips 102 and is directed through a separate aperture in assembly 430. Preferably, assembly 430 includes a central aperture 432 which communicates with a hollow mandrel 434. Stacked strips 420 from roller 410 pass through aperture 432 and through hollow mandrel 434. Stacked strips 422, 424, 426 and 428 from respective rollers 412, 414, 416 and 418 pass through peripheral apertures 442, 444, 446 and 448. Supply roller 410 is preferably maintained under tension, so as to maintain strips 102 supplied therefrom and via an auxiliary roller assembly 450 under tension.

As will be described hereinbelow in detail, strips 102 from roller 410 extend axially from assembly 430 along a longitudinal processing axis 460, extending parallel to the X-axis, under tension.

A first selectably closable clamp 462 is mounted onto chassis 402. A second selectably closable welding clamp 464 is mounted onto chassis 402 downstream of clamp 462 along processing axis 460 and preferably includes heating elements 465. A third clamp 466 is mounted on displaceable table assembly 404, downstream of clamp 464 along processing axis 460. A fourth clamp 468 is mounted on support assembly 408, downstream of clamp 466 along processing axis 460. A combined fifth clamp 470 and guillotine blade 472 are mounted on chassis 402, downstream of fourth clamp 468. Fifth clamp 470 includes a top clamp portion 474 and a bottom clamp portion 476 which also defines a blade engagement surface 480. Guillotine blade 472 is fixed to top clamp portion 474 and is displaced together therewith along a Z-axis as indicated by arrows 482. Blade engagement surface defining portion 480 is fixed to bottom clamp portion 476 and is displaced together therewith along a Z-axis as indicated by arrows 484.

Enlarged section I illustrates stacked strips 420, 422, 424, 426 and 428 extending through respective apertures 432, 442, 444, 446 and 448 in assembly 430. Enlarged section II and enlarged section III illustrate respective open and closed states of selectably closable clamp 462. Enlarged sections IV and V illustrate respective open and closed states of second selectably closable welding clamp 464. Enlarged sections VI and VII illustrate respective open and closed states of third clamp 466. Enlarged sections VIII and IX illustrate respective open and closed states of fourth clamp 468. Enlarged sections X, XI, XII and XIII illustrate four successive operational orientations of combined fifth clamp 470 and guillotine blade 472.

Clamp assembly 462 is arranged to intermittently clamp stacked outer biomass attachment strips 422, 424, 426 and 428 onto an outer surface of mandrel 434, as seen in enlarged section III, so as to prevent axial movement of strips 422, 424, 426 and 428 while stacked inner biomass attachment strips 420, extending through hollow mandrel 434 are free to be retracted by virtue of the tension applied thereto, thus producing outward bowing of outer biomass attachment strips 422, 424, 426 and 428.

Welding clamp 464 provides intermittent welding engagement of inner and outer biomass attachment strips 420, 422, 424, 426 and 428, as shown in enlarged section V, thereby joining stacked strips 420, 422, 424, 426 and 428 together at predetermined intervals along the lengths thereof. It is appreciated that such intervals are longer for outer biomass attachment strips 422, 424, 426 and 428, than for inner biomass attachment strips 420.

Combined fifth clamp 470 and guillotine blade 472 are operative to intermittently cut the intermittently joined biomass attachment strips 420, 422, 424, 426 and 428 at locations 490 where they are joined, thereby to provide individual, separate, compressible biomass carriers 100 whose ends 104 and 106 correspond to locations 490.

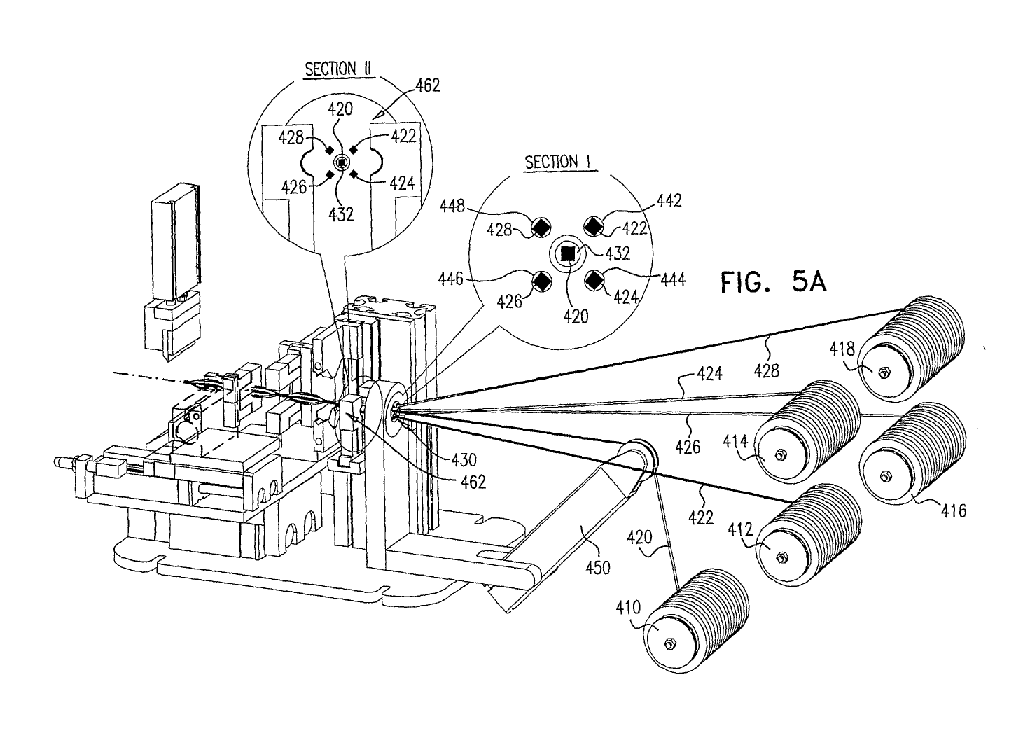

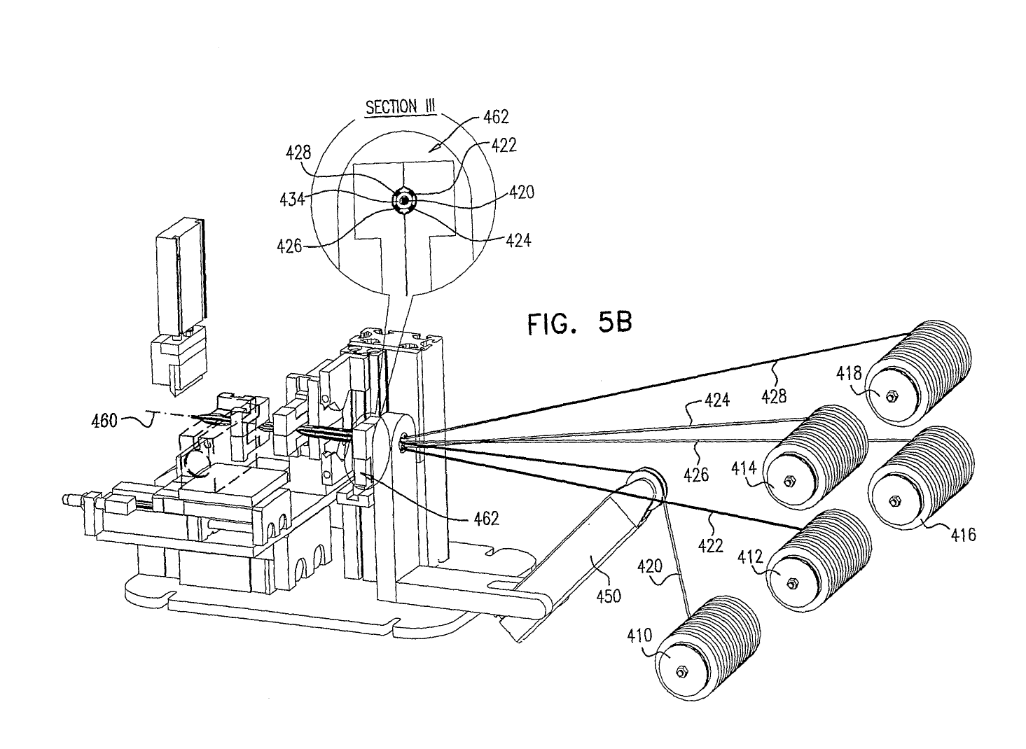

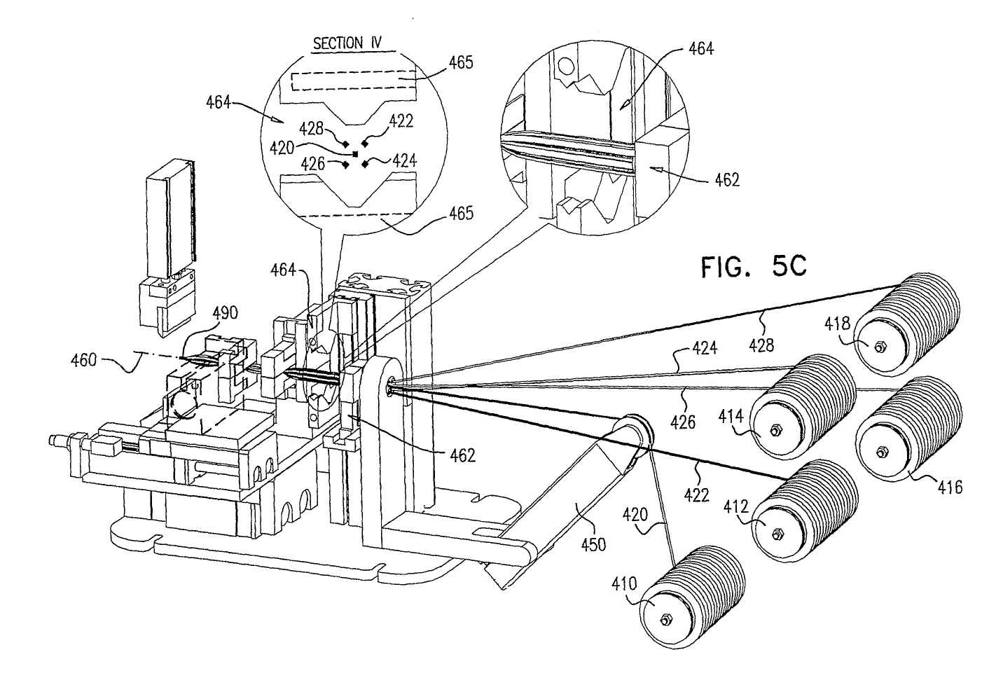

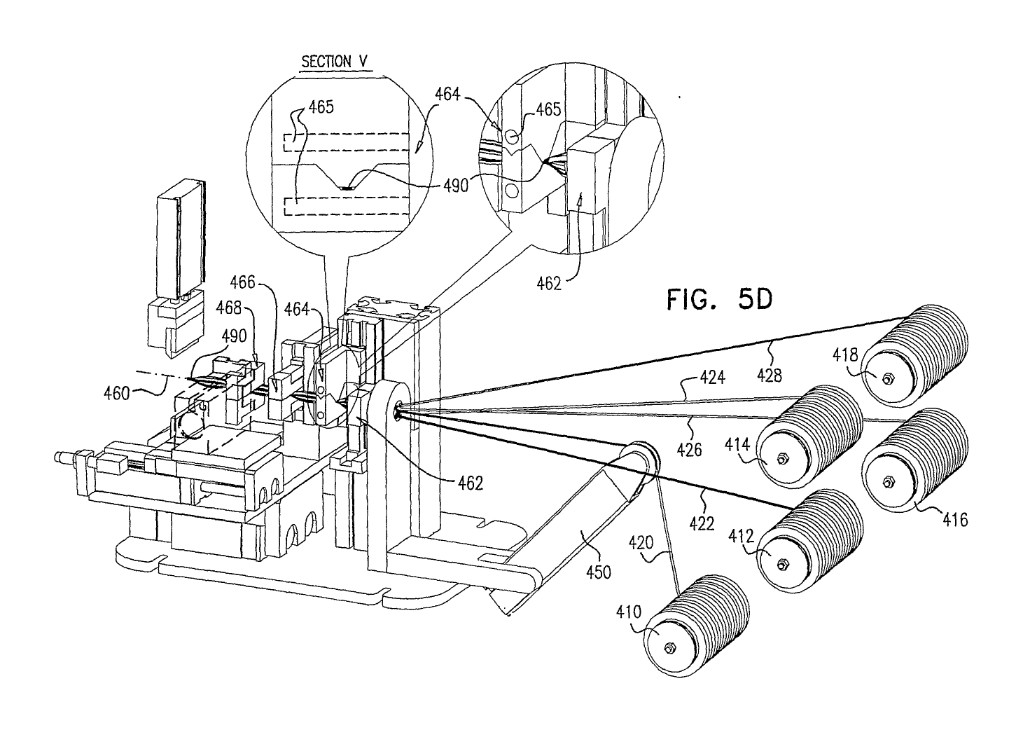

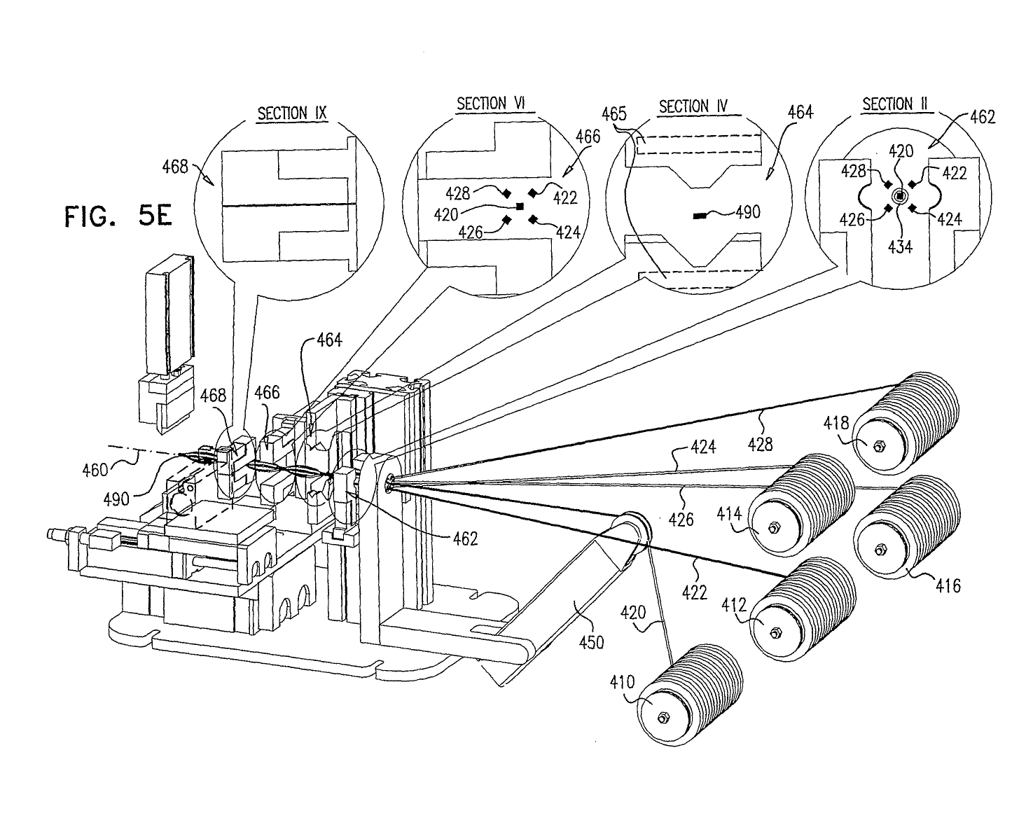

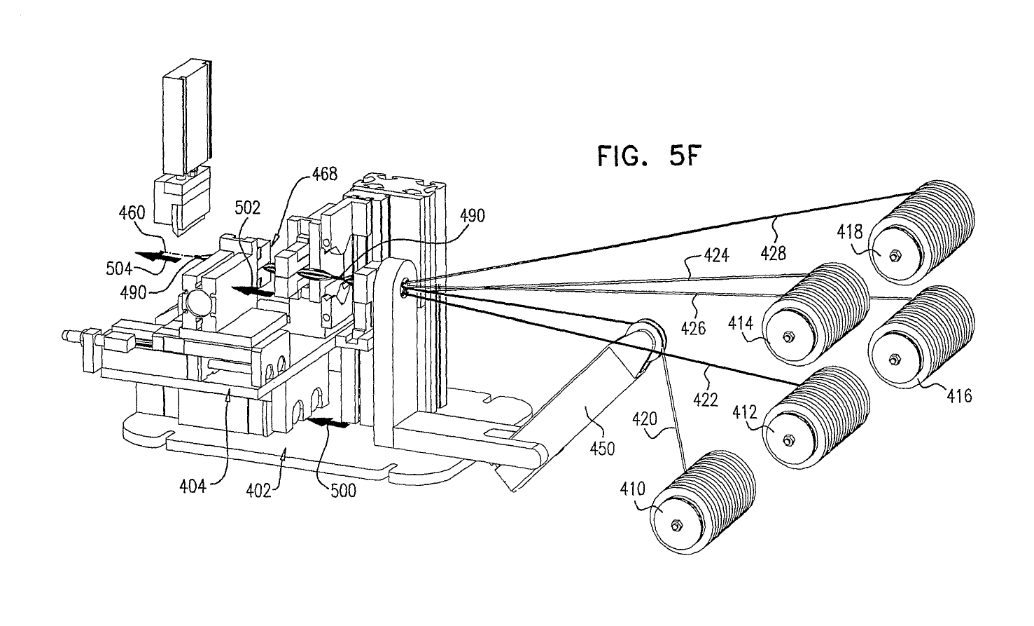

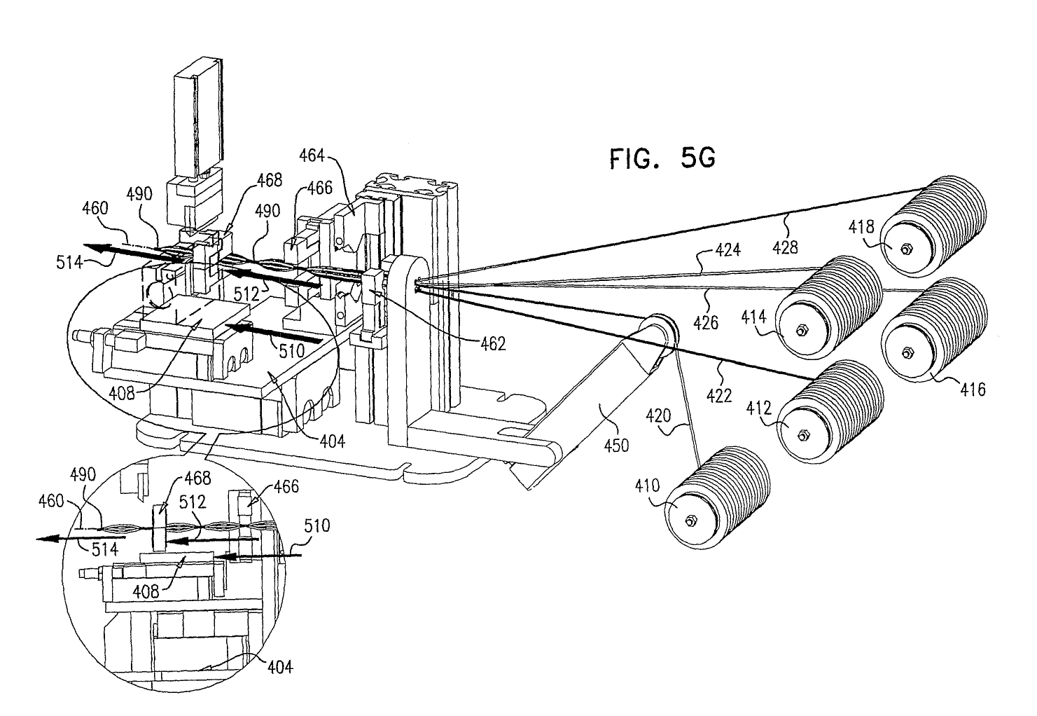

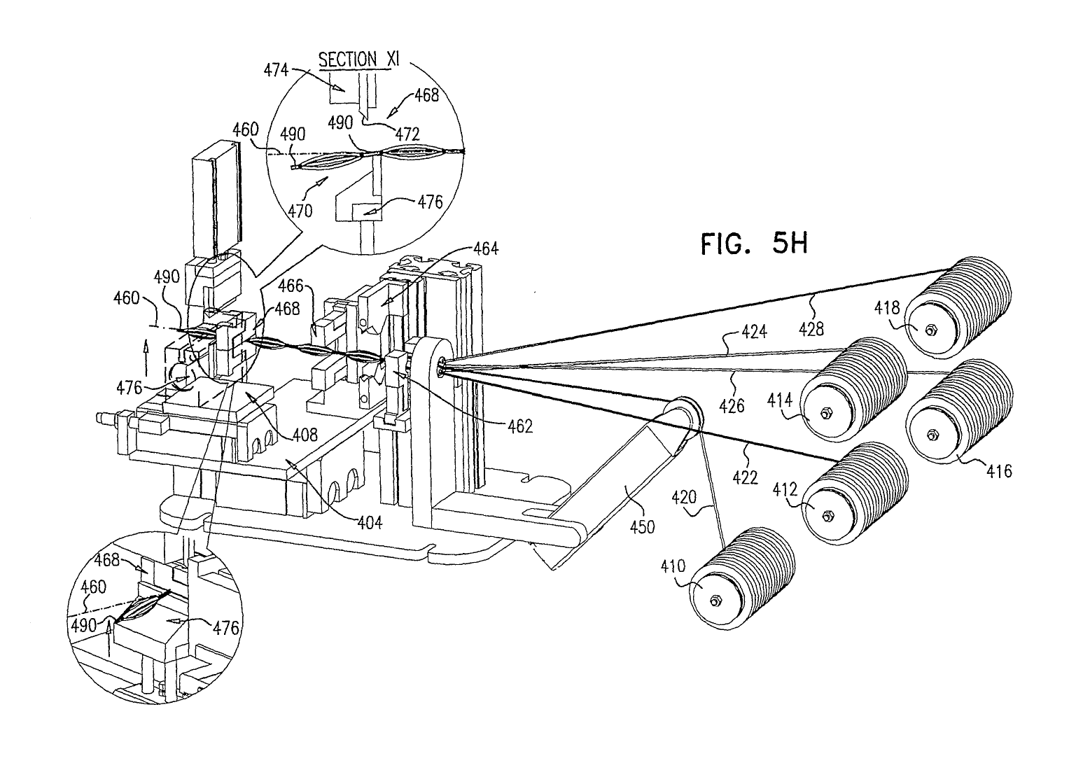

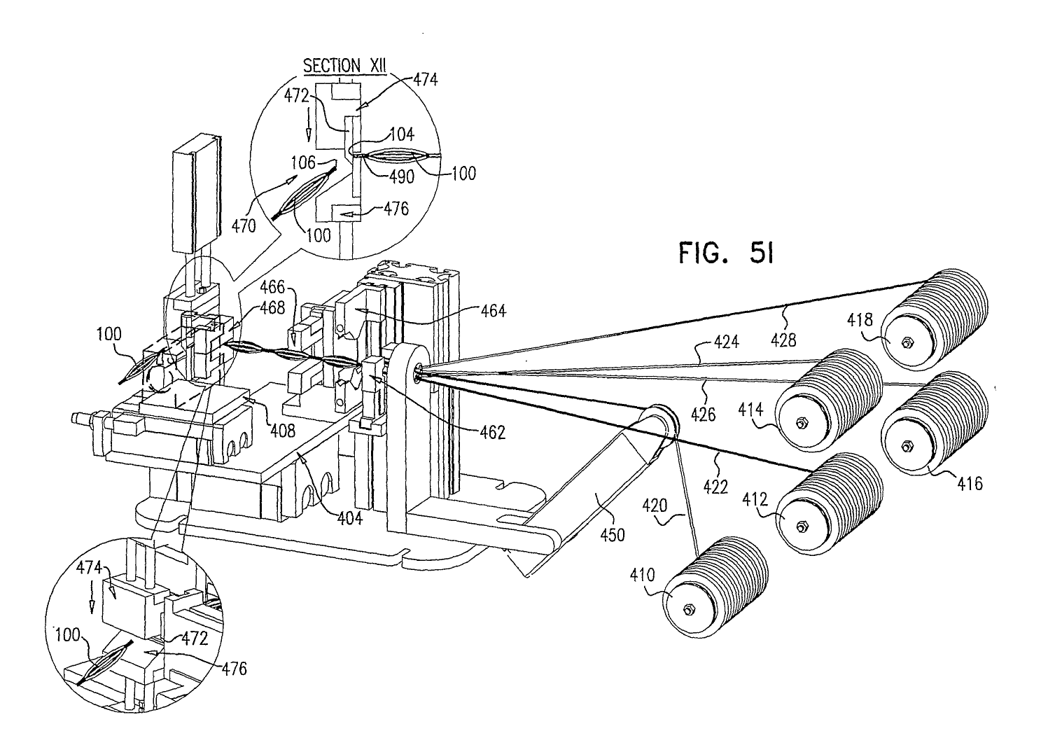

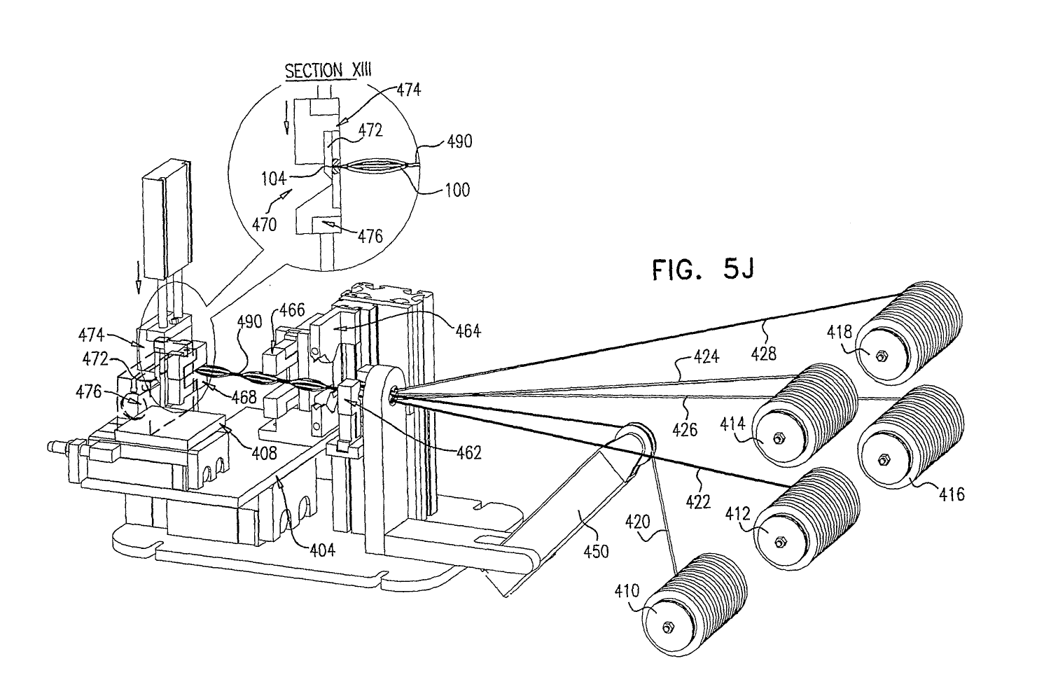

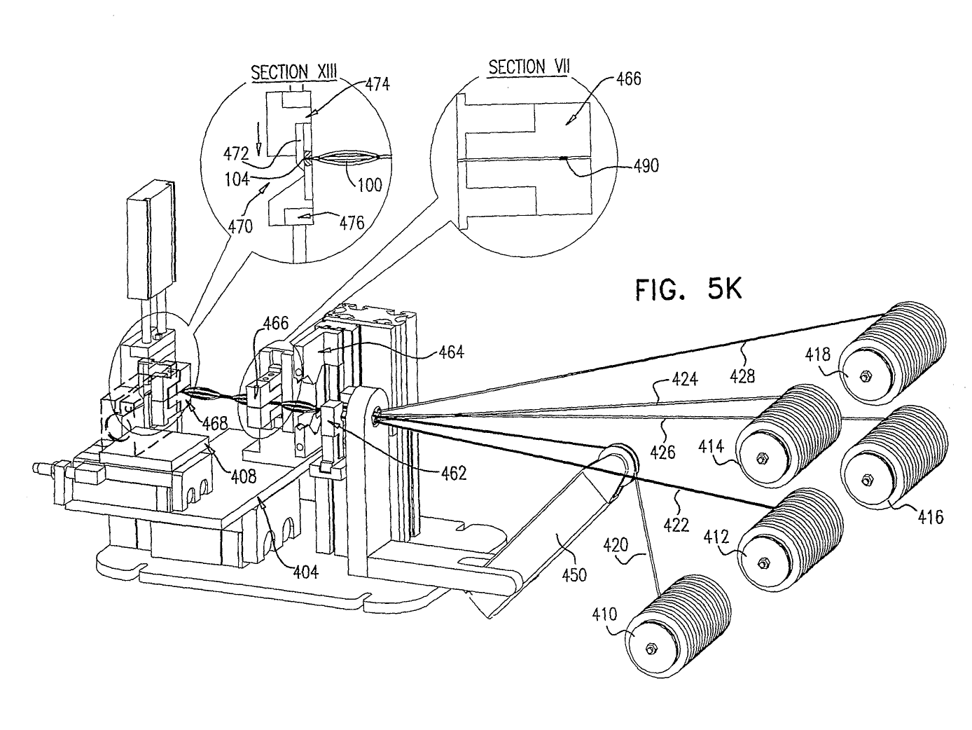

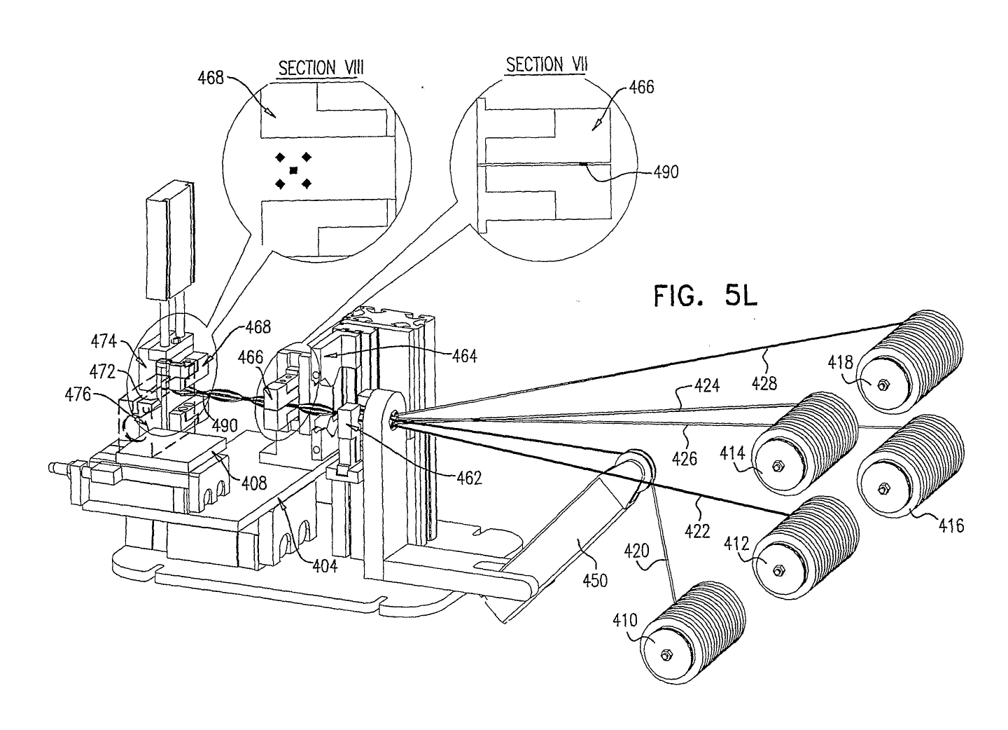

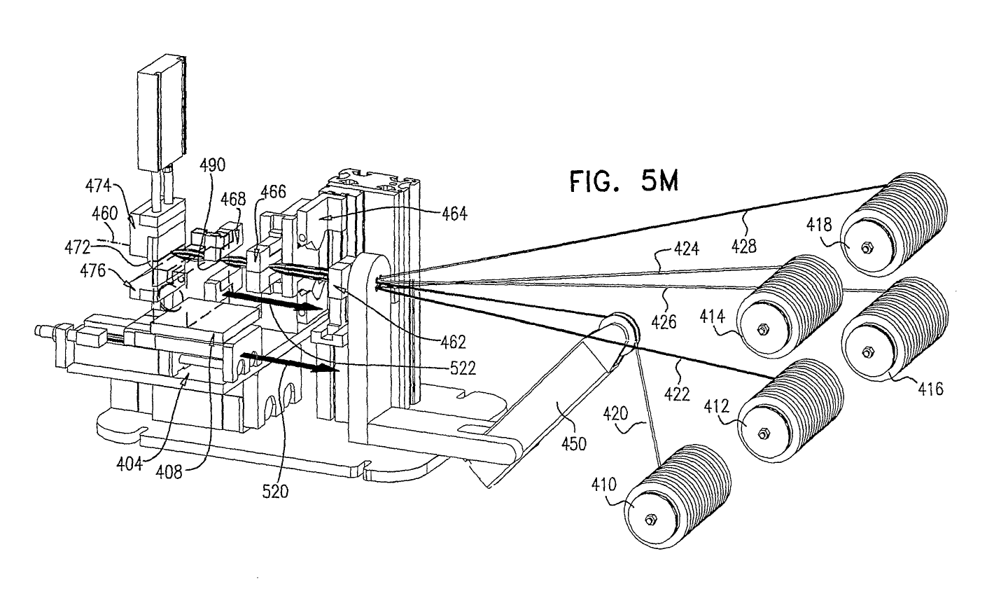

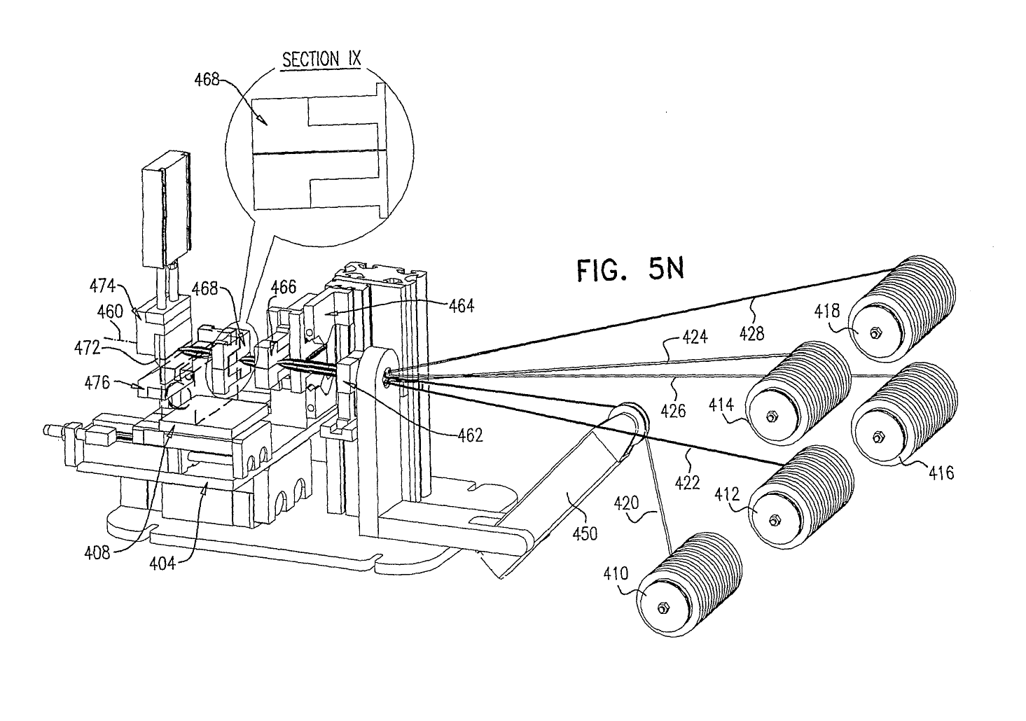

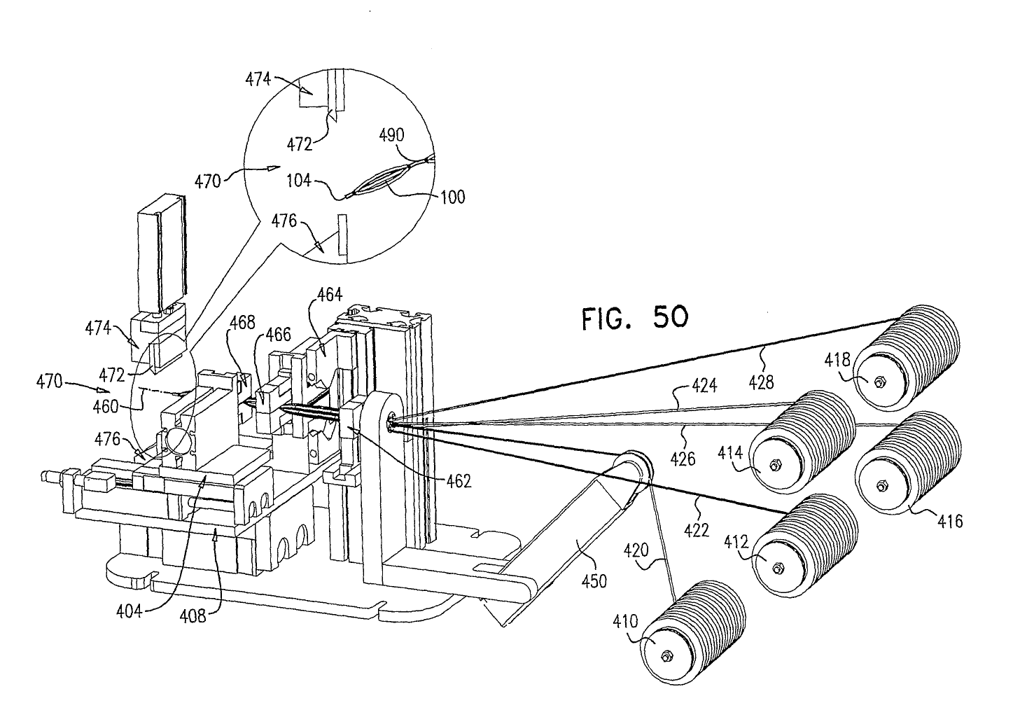

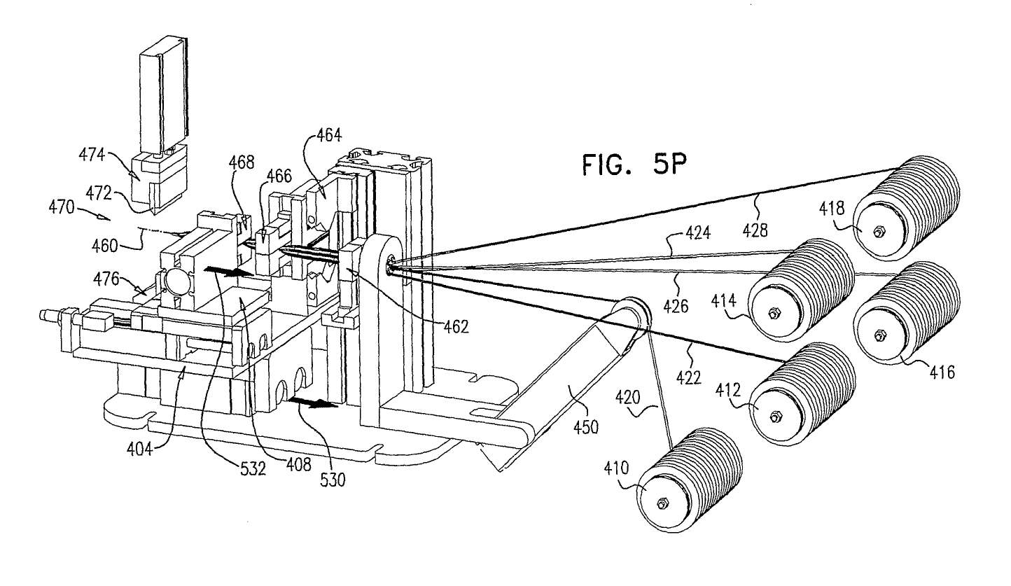

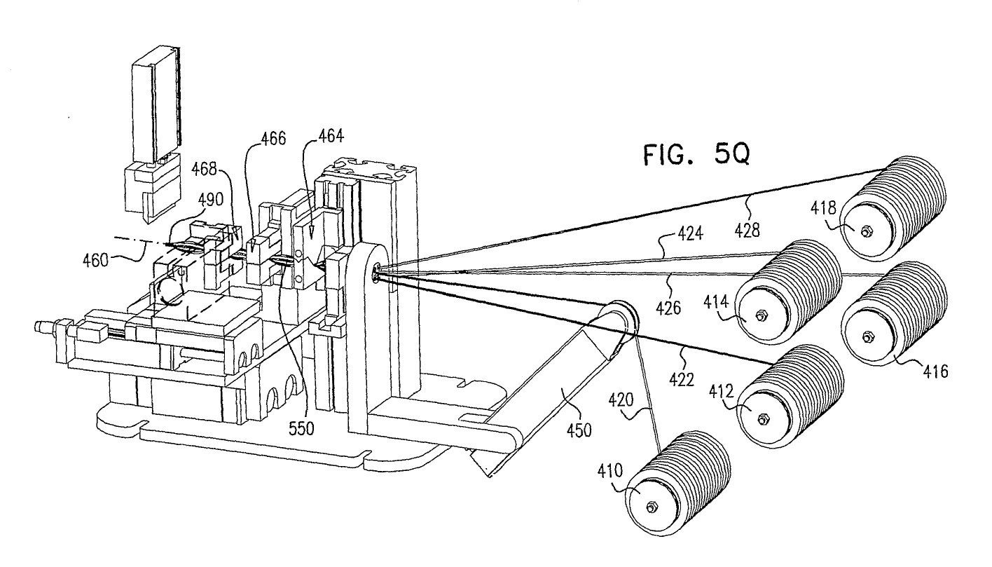

Reference is now made to FIGS. 5A-5Q, which illustrate generally sequential steps in the manufacture of a biomass carrier of the type shown in FIG. 1A using the apparatus of FIGS. 4A & 4B. It is appreciated that the manufacture is a continuous process and thus the explanation thereof which follows begins from a somewhat arbitrary starting point.

FIG. 5A shows the arrangement of stacked strips 420, 422, 424, 426 and 428 at assembly 430 (enlarged section I) wherein stacked strips 420 are under tension and clamp 462 is open (enlarged section II).

FIG. 5B shows clamp 462 closed (enlarged section III) such that stacked strips 422, 424, 426 and 428 are clamped against an outer cylindrical surface of mandrel 434 and are thus retained against axial movement along processing axis 460.

FIG. 5C shows welding clamp 464 open (enlarged section IV), while clamp 462 is closed, just prior to welding of stacked strips 420, 422, 424, 426 and 428 together at locations 490.

FIG. 5D shows welding clamp 464 closed (enlarged section V), thereby welding stacked strips 420, 422, 424, 426 and 428 together at locations 490. It is appreciated that alternatively ultrasonic or other welding may be employed.

FIG. 5E shows welding clamp 464 open (enlarged section IV) following welding of stacked strips 420, 422, 424, 426 and 428 together at locations 490. Additionally it is seen that at this stage clamps 462 (enlarged section II) and 466 (enlarged section VI) are open and clamp 468 (enlarged section IX) is closed.

FIG. 5F shows displacement of table 404 relative to chassis 402 along the X axis and processing axis 460 in a direction indicated by an arrow 500. This displacement is typically of length 3 mm. Displacement of table 404 as indicated by arrow 500 provides corresponding displacement of currently closed clamp 468 as indicated by an arrow 502 and corresponding forward motion of all of strips 420, 422, 424, 426 and 428, including portions thereof that are intermittently welded at locations 490 and the trailing unwelded extent thereof, along processing axis 460 as indicated by an arrow 504.

FIG. 5G shows displacement of support assembly 408 relative to table 404 along the X axis and processing axis 460 in a direction indicated by an arrow 510, in the same direction as arrow 500. This displacement is typically of length 50 mm and may but need not necessarily take place concurrently with the displacement described hereinabove with reference to FIG. 5F. Displacement of support assembly 408 as indicated by arrow 510 provides corresponding displacement of currently closed clamp 468 as indicated by an arrow 512 and corresponding forward motion of all of strips 420, 422, 424, 426 and 428, including portions thereof that are intermittently welded at locations 490 and the trailing unwelded extent thereof, along processing axis 460 as indicated by an arrow 514.

FIG. 5H shows upward movement along the Z-axis of bottom clamp portion 476 (enlarged section XI), thus raising the forward end of intermittently joined strips 420, 422, 424, 426 and 428 (enlarged section XI) to a generally horizontal orientation.

FIG. 5I shows downward cutting movement of guillotine blade 472, thereby cutting the intermittently joined strips 420, 422, 424, 426 and 428 at a location 490 thus forming a discrete detached biomass carrier 100 (FIGS. 1A and 1B) (enlarged section XII), thereby defining a back end 106 of discrete detached biomass carrier 100 and a forward end 104 of a next-to-be-detached biomass carrier 100.

FIG. 5J shows further downward movement of guillotine blade 472 and of top clamp portion 474, thereby clamping forward end 104 of next-to-be-detached biomass carrier 100 (enlarged section XIII) and enabling tension to be maintained of all of strips 420, 422, 424, 426 and 428, including portions thereof that are intermittently welded at locations 490 and the trailing unwelded extent thereof.

FIG. 5K shows closing of clamp 466 (enlarged section VII) which takes place while forward end 104 of next to be detached biomass carrier 100 is clamped (enlarged section XIII).

FIG. 5L shows opening of clamp 468 (enlarged section VIII) while clamp 466 is closed (enlarged section VII).

FIG. 5M shows displacement of support assembly 408 relative to table 404 along the X axis and processing axis 460 in a direction indicated by an arrow 520, opposite to arrows 500 and 510. This displacement is typically of length 50 mm. Displacement of support assembly 408 as indicated by arrow 520 provides corresponding displacement of currently open clamp 468 as indicated by an arrow 522 but does not provide corresponding motion of any of strips 420, 422, 424, 426 and 428, including portions thereof that are intermittently welded at locations 490 and the trailing unwelded extent thereof, along processing axis 460, which are currently clamped against axial movement along processing axis 460 by clamp 470.

FIG. 5N shows clamp 468 closed (enlarged section IX).

FIG. 5O shows disengagement of combined fifth clamp 470 and guillotine blade 472 from forward end 104 of next to be detached biomass carrier 100.

FIG. 5P shows displacement of table 404 along the X axis and processing axis 460 in a direction indicated by an arrow 530, opposite to arrows 500 and 510. This displacement is typically of length 3 mm. Displacement of table 404 as indicated by arrow 530 provides corresponding displacement of currently closed clamp 468 as indicated by an arrow 532. At this stage, clamp 462 is closed, thus clamping stacked strips 422, 424, 426 and 428 to mandrel 434 (not shown), but not clamping stacked strips 420. As a result rearward displacement of table 404 produces outward bowing of stacked strips 422, 424, 426 and 428 but not of stacked strips 420, which are not clamped and which are under tension.

FIG. 5Q shows closing of welding clamp 464, thereby welding together stacked strips 420, 422, 424, 426 and 428, when stacked strips 422, 424, 426 and 428 are in an outwardly bowed orientation and stacked strips 420 are not and thus fixing this orientation in a precursor biomass carrier, here designated by reference numeral 550.

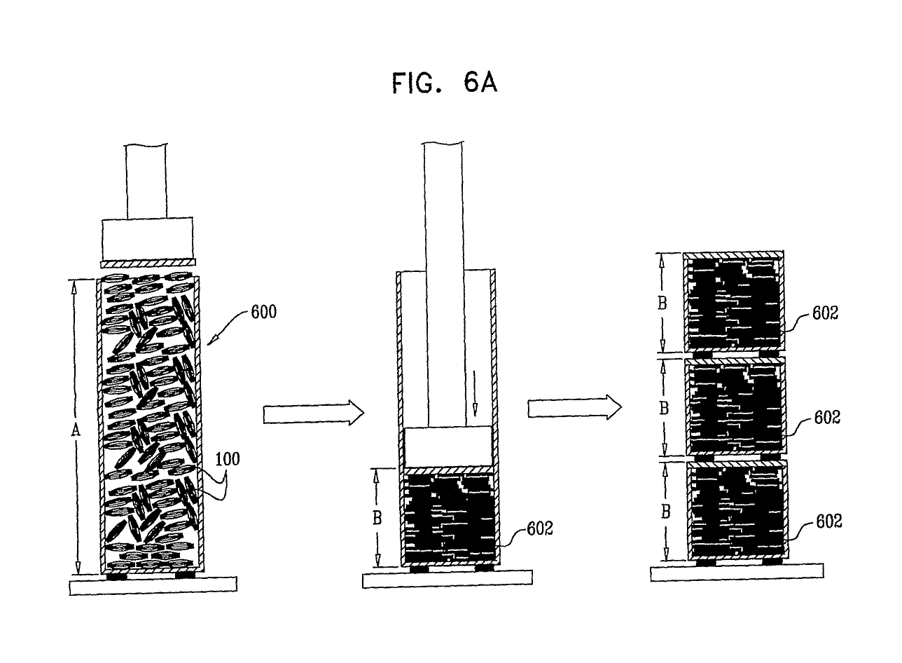

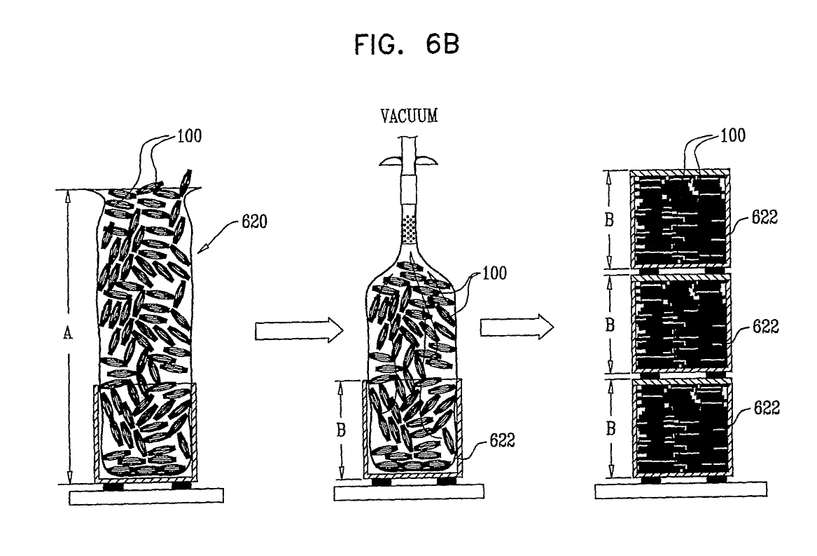

Reference is now made to FIG. 6A, which is a simplified illustration of compression of the biomass carriers of the type shown in FIG. 1A in accordance with a preferred embodiment of the present invention. In a typical situation, a plastic bag 600 of dimensions 1 meter×1 meter×3 meters, containing approximately 100,000 biomass carriers 100 in an uncompressed state, is compressed using positive pressure by a factor of approximately 4, to a package 602 having a size of approximately 1 meter×1 meter×1 meter.

Reference is now made to FIG. 6B, which is a simplified illustration of compression of the biomass carriers of the type shown in FIG. 1A in accordance with another preferred embodiment of the present invention. In a typical situation, a plastic bag 620 of dimensions 1 meter×1 meter×3 meters, containing approximately 100,000 biomass carriers 100 in an uncompressed state, is compressed under vacuum by a factor of approximately 3, to a package 622 having a size of approximately 1 meter×1 meter×1 meter.

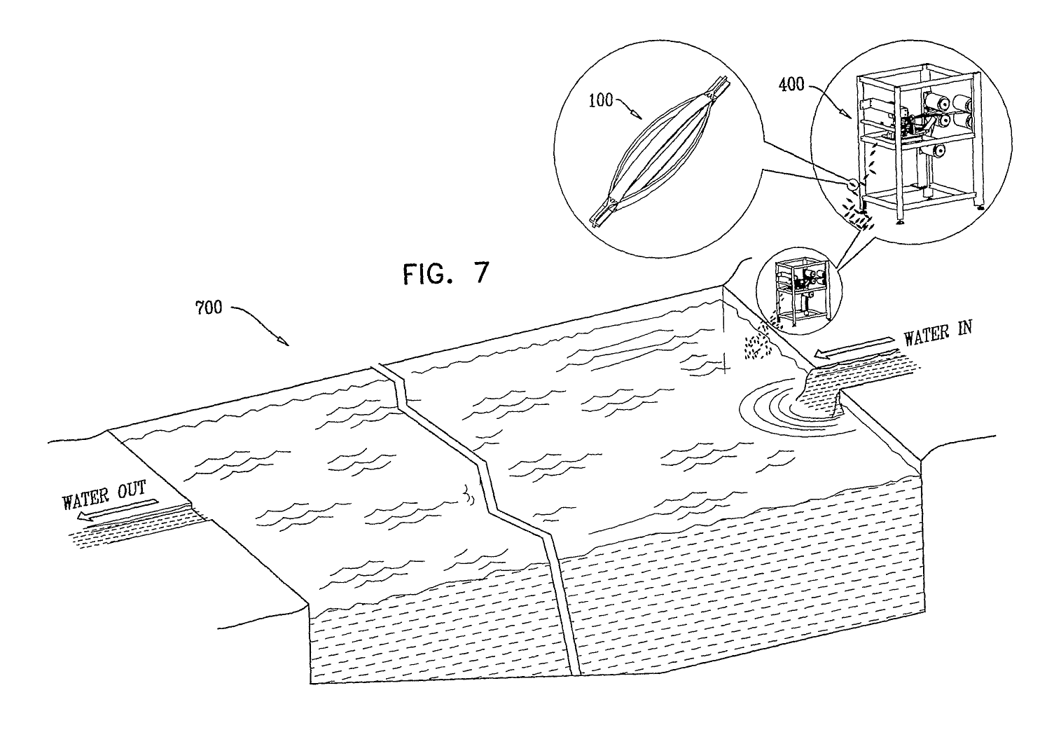

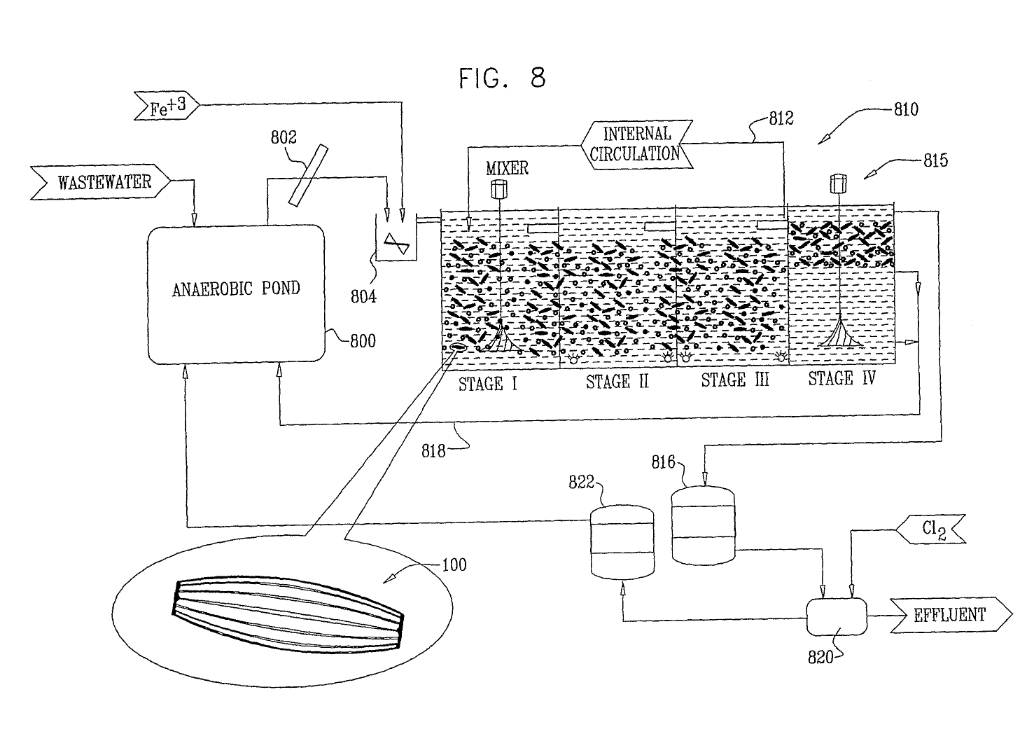

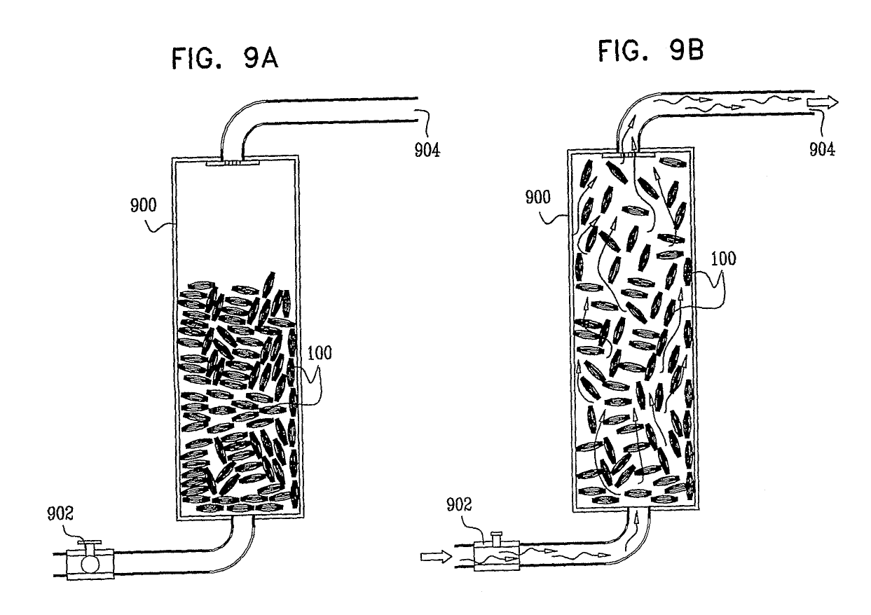

Reference is now made to FIG. 7, which illustrates on site manufacture of biomass carriers in accordance with a preferred embodiment of the present invention. As seen in FIG. 7, apparatus 400 for manufacture of biomass carriers 100 (FIG. 1A) is preferably located at a fluid treatment site, such as a water treatment facility 700. Fluid treatment site may be any suitable fluid treatment site which employs biomass carriers. Examples of fluid treatment facilities of this type are described hereinbelow with reference to FIGS. 8-9B and in assignee's patents and patent applications U.S. Pat. No. 6,616,845; U.S. Pat. No. 6,726,838; Published PCT Patent Application No. WO 02096806; Published PCT Patent Application No. WO 2007029256 and Published PCT Patent Application No. WO 2008018077 referenced hereinabove, the disclosures of which are hereby incorporated by reference.

Preferably, apparatus 400 is operative to dispense biomass carriers directly into a biogenerator or other receptacle as they are being manufactured, thereby realizing substantial savings in the cost of storage and transportation of the biomass carriers.

Reference is now made to FIG. 8, which is a simplified illustration of a wastewater treatment system constructed and operative in accordance with a preferred embodiment of the present invention.

Water, such as municipal wastewater, is preferably supplied to an anaerobic pond 800 for flow equalization and suspended solids precipitation, and, following settlement of some solids therefrom, is optionally and preferably supplied, preferably via a screen 802, to a sulfide precipitation tank 804, which also receives a supply of Fe⁺³ ions, preferably in the form of an aqueous solution. Downstream of sulfide precipitation tank 804, the water is supplied to a multistage media based water treatment subsystem 810, typically including four stages, here identified as stages I, II, III and IV.

Stage I is preferably an anoxic pre-denitrification stage, while stages II and III are aerobic stages. Internal circulation is preferably provided from stage III back to stage I, as indicated at reference numeral 812. Circulation of activated sludge is preferably avoided in water treatment subsystem 810.

Stages I, II and III of the multistage wastewater treatment subsystem 810 are preferably of the type described in applicant/assignee's U.S. Pat. No. 6,616,845 and Published PCT Patent Application No. WO 2008018077, the disclosures of which are hereby incorporated by reference, which employs biomass carriers, preferably of the type described herein and more preferably of the type illustrated in FIG. 1A.

Partially treated water from stage III of multistage wastewater treatment subsystem 810 is supplied to a denitrification and clarification stage 815, which preferably forms stage IV and includes a layer of biomass supporting media, preferably biomass carriers 100 (FIG. 1A) near to or at the top of thereof. The denitrification and clarification stage 815 produces denitrified and clarified water which may be utilized following further mechanical filtration in a filtration stage 816. Sludge from the denitrification and clarification stage 815 is preferably supplied to the anaerobic pond 800, as indicated by reference numeral 818. Alternatively, the sludge may be treated by any other conventional sludge treatment process.

Downstream of filtration stage 816, the denitrified and clarified water is supplied to a final treatment stage 820, at which chlorine treatment may be applied for disinfecting according to local regulations.

Water from final treatment stage 820 is preferably used to periodically backwash part or all of the filters from solids accumulated therein, as indicated at reference numeral 822. The backwash water from the filters is preferably discharged into pond 800 for the purpose of solids precipitation and stabilization.

Reference is now made to FIGS. 9A and 9B, which illustrate a gas treatment system constructed and operative in accordance with a preferred embodiment of the present invention. As seen in FIG. 9A, there is provided a bioreactor 900 including an inlet 902 and an outlet 904. The bioreactor 900 contains, but is preferably not entirely filled with, biomass carriers 100 (FIG. 1A).

As seen in FIG. 9B, gas to be treated, such as contaminated air, is supplied to inlet 902 of bioreactor 900 and causes movement of biomass carriers 100 therein, thus resulting in a limited amount of desirable sloughing of biomass therefrom and purifying the contaminated air. Purified air leaves the bioreactor 900 via outlet 904.

It will be appreciated by persons skilled in the art that the present invention is not limited to what has been particularly shown and described hereinabove. Rather, the scope of the invention includes both combinations and sub-combinations of various features described hereinabove as well as modifications and variations thereof which would occur to a person skilled in the art upon reading the foregoing description and which are not in the prior art.

1. A biomass carrier comprising:
a multiplicity of generally elongate plastic biomass attachment elements joined to each other at least one location therealong, said multiplicity of generally elongate plastic biomass attachment elements being mutually arranged to define a generally deformable biomass carrier including generally outer disposed biomass attachment elements and generally inner disposed biomass attachment elements.

2. A biomass carrier according to claim 1 and wherein said multiplicity of generally elongate plastic biomass attachment elements has a ratio of effective biomass attachment surface area to weight of between 10 to 30 m²/Kg.

3. (canceled)

4. A biomass carrier according to claim 1 and wherein said multiplicity of generally elongate plastic biomass attachment elements has a ratio of effective biomass attachment surface area to overall volume of between 600 to 1500 m²/m³ carriers.

5. (canceled)

6. A biomass carrier according to claim 1 and wherein said multiplicity of generally elongate plastic biomass attachment elements includes generally elongate plastic biomass attachment elements having at least two different lengths, joined to each other at least at one location.

7-10. (canceled)

11. A method for manufacturing biomass carriers, the method comprising:
providing generally elongate plastic biomass attachment elements; and
joining said generally elongate plastic biomass attachment elements at at least one location therealong, thereby to define generally deformable biomass carriers.

12-16. (canceled)

17. A method for manufacturing biomass carriers according to claim 11 and wherein at least said joining step is carried out in propinquity to a fluid treatment system.

18. A method for manufacturing biomass carriers according to claim 17 and also comprising dispensing multiple ones of said biomass carriers into said fluid treatment system as they are manufactured.

19. A method for manufacturing biomass carriers according to claim 11 and also comprising:
compressing multiple ones of said biomass carriers;
shipping said multiple ones of said biomass carriers following compression thereof to a fluid treatment system site; and
decompressing said multiple ones of said biomass carriers at said fluid treatment system site.

20. (canceled)

21. Apparatus for manufacturing biomass carriers, the apparatus comprising:
a positioner for positioning a multiplicity of generally elongate plastic biomass attachment elements; and
a binder, joining said plurality of generally elongate plastic biomass attachment elements downstream of said positioner at least one location therealong, thereby to define generally deformable biomass carriers.

22-26. (canceled)

27. Apparatus for manufacturing biomass carriers according to claim 21 and wherein at least said binder is located in propinquity to a fluid treatment system.

28. Apparatus for manufacturing biomass carriers according to claim 27 and also comprising a dispenser, dispensing multiple ones of said biomass carriers into said fluid treatment system as they are manufactured.

29. Apparatus for manufacturing biomass carriers according to claim 21 and also comprising a compressor, compressing multiple ones of said biomass carriers.

30. (canceled)

31. A fluid treatment system comprising:
at least one bioreactor having a fluid inlet for receiving fluid to be treated and a fluid outlet for providing treated fluid; and
a plurality of biomass carriers located within said at least one bioreactor, at least some of said plurality of biomass carriers each comprising a multiplicity of generally elongate plastic biomass attachment elements joined to each other at least one location therealong, said multiplicity of generally elongate plastic biomass attachment elements being mutually arranged to define a generally deformable biomass carrier including generally outer disposed biomass attachment elements and generally inner disposed biomass attachment elements.

32. A fluid treatment system according to claim 31 and wherein said bioreactor operates in at least one of an aerobic, an anoxic and an anaerobic mode of operation.

33. A fluid treatment system according to claim 31 and wherein said bioreactor is operative for treating a liquid.

34-36. (canceled)

37. A fluid treatment method comprising:
providing a bioreactor having located therewithin a plurality of biomass carriers, at least some of said plurality of biomass carriers each comprising a multiplicity of generally elongate plastic biomass attachment elements joined to each other at least one location therealong, said multiplicity of generally elongate plastic biomass attachment elements being mutually arranged to define a generally deformable biomass carrier;
receiving at a fluid inlet of said bioreactor, fluid to be treated; and
providing treated fluid at a fluid outlet of said bioreactor.

38. A fluid treatment method according to claim 37 and wherein said bioreactor operates in at least one of an aerobic, an anoxic and an anaerobic mode of operation.

39. A fluid treatment method according to claim 37 and wherein said bioreactor is operative for treating a liquid.

40. A fluid treatment method according to claim 37 and wherein said bioreactor is operative for treating a gas.

41-42. (canceled)
